(19) United States
(12) Patent Application Publication
(10) Pub. No.: US 2011/0054484 A1
(43) Pub. Date: Mar. 3, 2011

(54) MINIMALLY INVASIVE ENDOSCOPIC SYSTEMS FOR PLACING INTRAMEDULLARY NAILS AND METHODS THEREFOR

(76) Inventor: Mark Leonard Brandon, Staten Island, NY (US)

(21) Appl. No.: 12/745,561

(22) PCT Filed: Dec. 30, 2009
(86) PCT No.: PCT/US09/69832

§ 371 Date: Jun. 1, 2010

Related U.S. Application Data
(60) Provisional application No. 61/141,346, filed on Dec. 30, 2008.

(51) Int. Cl. A61B 17/56

(52) U.S. Cl. 606/96

(57) ABSTRACT

A system for percutaneously placing intramedullary nails includes a guide wire (20) percutaneously insertable into tissue (T) for forming an opening in the tissue and engaging a cortex of a bone (FB), at least one tissue dilator (40A) slideable over an outer surface of the guide wire (20) for widening the tissue opening formed by said guide wire, and a cannula (60) slideable over an outer surface of said at least one tissue dilator (40A) for further widening the tissue opening and providing access to the cortex of the bone. The cannula (60) has an outer wall (66) extending between proximal and distal ends thereof, the outer wall having a smaller portion including a door (76) that extends between the proximal end distal ends of the cannula and a larger portion that bounds the door. The door (76) is selectively detachable from the larger portion of the outer wall (66) for opening an elongated slot (74) in the outer wall that extends from the proximal end to the distal end of the cannula.

### CROSS-REFERENCE TO RELATED APPLICATIONS

The present application claims the benefit of U.S. Provisional Application Ser. No. 61/141,346, filed Dec. 30, 2008, entitled “Endoscopic Minimally Invasive System for Placement of Intramedullary Nails,” the disclosure of which is hereby incorporated by reference herein.

### BACKGROUND OF THE INVENTION

1. Field of the Invention

The present invention generally relates to systems, devices and methods for treating bone fractures and more specifically relates to systems, devices and methods for treating fractures in long bones using intramedullary nails.

2. Description of the Related Art

Vertebrates maintain their upright posture and resist the earth's gravitational pull with the aid of their bony structure. Vertebrates' bones are formed of a hard outer cortex that surrounds a soft, spongy cancellous portion. The cortical portion of the bone is stiffer than cancellous portion. In a bony structure, the long bones are those bones that are present in the limbs, such as the femur, tibia, fibula, humerus, radius and ulna. In the skeletal system, the flat bones are those bones that make up the axial skeleton in the trunk and cranium, such as the pelvis and the skull.

When a break or fracture occurs in a long bone the separated portions of the long bone must be held together in close apposition while healing occurs. Over the years, there have been many different techniques used for treating fractures of the long bone and to help keep the separated portions of the long bones in close contact with one another in order to aid in the healing of these fractures. In ancient Egyptian times, several methods of external splinting were utilized for treating long bone fractures. Up until the early 20^{th} century, fracture patients were often treating with casting, splinting, or bed traction for prolonged periods, which frequently resulted in deformities, disabilities, delayed healing, non-healing, amputation, and death.

Fractures of long bones may be treated with open reduction and internal fixation, which involves making an incision through the soft tissue overlying the affected long bone and placing a metallic plate and screws on either side of the fracture to hold the fracture fragments in alignment that will enable fracture healing. One disadvantage of this fracture treatment method is that the incision through the soft tissue may result in more pain, nerve injury, blood vessel injury, necrosis (death) of the affected bone or muscles around the fractured long bone, unsightly scarring, wound infection, and/or a weakened bone by a process called stress shielding whereby the normal forces that are placed across a bone via walking or lifting objects are passed through the metallic plate as opposed to the bone. As a result, less dense bone may be prone to further subsequent fractures. Long bone fractures may also be treated with an external fixation device whereby metallic screws are placed into the long bone on either side of the fracture and connected to a series of bars external to the skin to hold the fractured bony fragments in an adequate position to enable subsequent bone healing. The disadvantages to this method of fracture treatment include loss of reduction of the fractured bone fragments so that they move into an unacceptable position, delayed healing, pin site infections, and joint stiffness.

In the 1930's, the Smith-Peterson nail was introduced whereby the nail was inserted into the intramedullary canal of the femur for the treatment of femoral fractures. The Smith-Peterson nail enabled immediate fixation of femoral fractures and earlier mobilization of the patient. In addition, the intramedullary nailing technique for the treatment of femoral fractures resulted in a decrease in the morbidity and mortality of patients. A number of other nails have been introduced for fixing fractures of long bones including the jewett nail, Enders rods, the Gamma Nail, the T2 tibial and humeral nails.

Intramedullary nails may be placed down the entire length of long bones for added fixation and to prevent displacement of potential future fractures in these long bones in patients prone to factures by conditions ranging from bone metastases in cancer patients to patients with weakened bone due to bone disease such as osteoporosis. Bone screws or cannulated bone screws may be inserted through the intramedullary nails on either side of a fracture or across the fracture site to prevent distraction, collapse or rotation across the fracture site of the long bone. The earlier design convention intramedullary nails did not provide these bones screws or cannulated bone screws to go through the intramedullary nail. The advantages of intramedullary nails over other methods of fracture treatment include earlier mobilization, earlier fracture healing, better maintenance of fracture reduction or aligning, smaller incisions, less incisional pain, smaller scars, decreased pain with ambulation, and load sharing across the fracture site such that the forces along the bone pass through both the intramedullary nail and through the bone resulting in less stress shielding than is seen with open reduction and plate fixation of long bones and a more dense bone that is less likely to subsequently fracture than those treated with plate fixation.

Recent advances in intramedullary nailing devices include the use of an external targeting guide that facilitates the placement of the proximal, distal and lag screws through the intramedullary nail. There also exists expanding devices that expand after placement in the intramedullary canal of long bones to provide a tight fit and help prevent rotation or collapse at the fracture site. These advances are disclosed in U.S. Pat. No. 5,057,103 to David, U.S. Pat. No. 4,632,101; U.S. Pat. No. 4,862,883; and U.S. Pat. No. 4,721,103 to Freedland, U.S. Pat. No. 6,077,264 to Chemello, U.S. Pat. No. 4,590,930 to Kurth, U.S. Pat. No. 4,453,539 to Raftopoulos, and U.S. Pat. No. 4,236,512 to Aginski.

The major disadvantage of these methods of fixation is that the nails are difficult to remove in the event of hardware device failure or infection. These methods also do not include the use of screws that pass through a long bone's cortex, which provides strong bone purchase and helps maintain the intramedullary construct in the ideal, selected position. This method of cortical screw passage through the intramedullary nail and the through the bone cortex was proposed previously in U.S. Pat. No. 4,275,717 to Bolesky. Further advances were made that enabled compression across the facture site with the aid of a compression screw that enables the lag screw that crosses the facture site (U.S. Pat. No. 6,443,954 to Bramlet).

In the 1990's, Howmedica of Rutherford, N.J. produced the “Gamma Nail” for the treatment of proximal femoral fractures through the intertrochanteric or subtrochanteric regions of the proximal femur. The first generation of the Gamma Nail was found to be too wide in diameter, especially at its distal end. This exaggerated width was determined to play a role in the increased incidence of pen-prosthetic fractures seen at the distal tip of intramedullary nail after it is inserted. The first generation of the Gamma Nail was also subsequently found to be too short for the treatment of subtrochanteric fractures of the femur. Since the inception of the first generation of the Gamma Nail, two more generations of the Gamma Nail have been developed and employ and more narrow distal tip and the Gamma Nail is now offered in longer lengths to allow for spanning the entire length of the femur. The Gamma Nail is the most commonly used intramedullary nail, although many other similarly intramedullary nails have been developed by several manufacturers for the treatment of not only femoral fractures, but for the treatment of fractures of other long bones. The intramedullary nails require several incisions through the skin, fat, fascia, and muscle and all soft tissue superficial to the long bone to allow for the placement of intramedullary nail, the proximal and distal locking screws, the lag screw and the end cap.

To date, there has been no utilization of fiber optic technology to assist in the direct visualization of the outer cortex of the long bone for the placement of an intramedullary nail. Some techniques involve using a fiber optic light cable that is placed inside the intramedullary nail itself for the purpose of illuminating the distal locking holes in the intramedullary nail to facilitate locating the distal locking holes, however, this does not involve using the fiber optic light source as a means to directly visualize the inside of the intramedullary nail nor does it utilize the fiber optic light source as a means of directly visualizing the bone cortex at the intramedullary nail insertion site. Moreover, the prior art does not utilize a fiber optic light source as a means to directly visualize the outside of the bone cortex at the site of the lag screw placement or at the placement of the distal locking screw. See U.S. Pat. Nos. 5,540,691 and 5,417,688 to Elstrom et al.

Despite the abovementioned advances in the operative treatment of patients suffering long bone fractures, these patients are still subjected to a lower activity level in the immediate post-operative period until the bone is sufficiently healed and the post-op physical rehabilitation is able to restore some of the muscle atrophy that occurs as a result of the relative inactivity. Due to the fact that these patients are unable to ambulate often in the few months post-operatively as they are accustom to, they are at increased risk of the potentially life-threatening blood clots, or deep vein thromboses, which may embolize to the lungs as pulmonary embolisms. Because of these potentially life-threatening complications associated with long bone fractures, these patients are usually placed on medications that prevent deep vein thromboses, such as enoxaprins, heparin, warfarin, and/or aspirin. The side effects of these medications include that the soft tissue incision where the intramedullary nails and the corresponding proximal locking screws, distal locking screws, and lag screws are placed will often bleed and/or drain for prolonged periods of time throughout the post-operative period. It is felt by many that this wound range is a necessary complication of intramedullary nail treatment of long bone fractures because the sharp soft tissue dissection with a scalpel or a similar device must be performed in order to place the intramedullary nail its corresponding components, the proximal and distal locking screws, and the lag screws. However, these deep, narrow incisions make it difficult if not impossible to obtain a good tight closure of the soft tissue layers traumatized by the scalpel. The trauma to the subcutaneous fat tissue caused by the sharp dissection with the scalpel for the placement of the intramedullary nail and its corresponding components may also cause some fat necrosis that can cause additional wound drainage. This wound drainage and fat necrosis caused by the sharp soft tissue dissection provides a dangers medium for the potential cultivation of bacteria and subsequent wound infection that may result in sepsis and death. If a wound infection should develop, this may result in osteomyelitis around the intramedullary nail and necessitate further surgery such as hardware removal and delayed fracture healing and delayed fracture treatment with a staged intramedullary nailing or even amputation. The associated wound infection may also result in sepsis and/or death. Other potential complications of the soft tissue dissection with a scalpel for the placement of the an intramedullary nail and its corresponding components include, but are not limited to, nerve laceration with subsequent motor paralysis, insensate extremities or painful neuromas, muscle lacerations with subsequent weakness, tendon laceration with subsequent immobility, blood vessel laceration with subsequent muscle necrosis or osteonecrosis of the affected bones with potential delayed or impaired fracture healing.

All the above noted side effects from sharp, scalpel dissection of soft tissue deep to the dermis may not only cause significant morbidity and mortality, but they may also necessitate further surgery and a subsequent morbidity and mortality risks.

The current surgical technique for intramedullary nail device placement rely heavily on the use of hazardous and carcinogenic x-ray radiation for visualization of the placement of the intramedullary nail, proximal and distal lock screws, lag screws and the end-cap. This x-ray radiation, in the form of fluoroscopic images, is harmful to the patient, the surgeon and all staff members in the operating room involved in the patient's care. There clearly exists a need for an intramedullary nailing system that minimizes not only the potentially harmful sharp soft tissue dissection utilized by all known prior intramedullary nailing systems, but a need also exists for an intramedullary nailing system that allows for some form of visualization of the hardware placement which minimizes or obviates the need for the use of harmful of x-ray radiation. The direct visualization of the starting placement of the intramedullary nail, which is enabled by this endoscopic, fiber optic, intramedullary nail placement system described herein, will help prevent inadequate fracture reduction and help prevent inadequate intramedullary nail placement that may occur with indirect fluoroscopically imaged intramedullary nail placement techniques currently being utilized. The endoscopic intramedullary nail devices listed herein enable the placement of intramedullary nails and all the necessary components of intramedullary nails including the proximal locking screws, the distal locking screws, the lag screws and the end-cap into long bones via methods described herein that would potentially offer the patient fracture healing with less soft tissue trauma, fewer complications, and better results that are superior to the methods of intramedullary nail placement currently being utilized.

In view of the foregoing, there is a need for percutaneous minimally invasive systems, devices and methods for the placement of intramedullary nails that obviates the need for sharp soft tissue dissection below the dermis.

### SUMMARY OF THE INVENTION

The present desirably provides an endoscopic system for placing intramedullary nails in long bones. The intramedullary nailing system disclosed herein preferably results in less trauma to the soft tissues caused by blunt dissection. In one embodiment, the present invention uses a series of tissue dilators so as to provide for gentle spreading of soft tissues as opposed to sharp soft tissue dissection, which may injury nerves, tendons and blood vessels in soft tissue between the skin and the long bone. The intramedullary nailing system of the present invention also preferably results in less bleeding and shorter surgical procedures than occur when using sharp soft dissection because the present invention obviates the need for suture repair in the soft tissue deep to the dermis. The present invention potentially also preferably lowers the risk of deep vein thromboses, lowers the risk of intra-operative myocardial infarction (heart attack) and lowers mortality risk due to the shortened surgical procedure time. In one embodiment, the present invention also improves healing of the soft tissue deep in the dermis due to the decreased trauma to the soft tissue, improves positioning of the guide wires and the intramedullary nail and all corresponding hardware devices due to the ability to directly visualize and select the optimal long bone cortical entry site of the guides wires and the intramedullary nail and all corresponding devices. The present invention also decreases the likelihood of hardware failure due to improved hardware positioning in the affected long bone, potentially decreases the risk of post-operative wound drainage due to decreased trauma to the soft tissues and decreased risk of traumatizing blood vessels in the soft tissue, and potentially decreases risk of wound infection, osteomyelitis, sepsis, septic shock and death associated with wound drainage.

In one embodiment, the present invention also preferably decreases the risk of secondary procedures for removal of hardware and subsequent hardware replacement associated with wound infection. The present invention also desirably results in less exposure to carcinogenic fluoroscopic radiation to the patient, the surgeon and the entire operating team.

In one embodiment, a system for percutaneously placing intramedullary nails includes a guide wire percutaneously insertable into tissue for forming an opening in the tissue and engaging a cortex of a bone, at least one tissue dilator slideable over an outer surface of the guide wire for widening the tissue opening formed by the guide wire, and a cannula slideable over an outer surface of the at least one tissue dilator for further widening the tissue opening and providing access to the cortex of the bone. The cannula desirably has an outer wall extending between proximal and distal ends thereof, the outer wall having a smaller portion including a door that extends between the proximal end distal ends of the cannula and a larger portion that bounds the door. The door is selectively detachable from the larger portion of the outer wall for opening an elongated slot in the outer wall that extends from the proximal end to the distal end of the cannula.

In one embodiment, the outer wall of the cannula has an inner surface and an outer surface, and the elongated slot in the outer wall extends from the inner surface of the outer wall to the outer surface of the outer wall. In one embodiment, the outer wall of the cannula has a cylindrical shape.

In one embodiment, the door on the cannula is preferably slideable toward the distal end of the cannula for closing the elongated slot in the outer wall and is slideable toward the proximal end of the cannula for opening the elongated slot in the outer wall. The system may include a locking system having a locked state that prevents the door from sliding toward the proximal end of the cannula and an unlocked state that releases the door for sliding toward the proximal end of the cannula. The locking system desirably includes a moveable locking element adapted to engage the sliding door when in the locked state and be disengaged from the sliding door in the unlocked state.

In one embodiment, the system preferably includes an intramedullary nail insertable into the cannula for passing through the tissue opening and engaging the cortex of the bone, and an alignment device coupled with the intramedullary nail. In one embodiment, while the intramedullary nail engages the bone, the cannula is moveable away from the intramedullary nail by detaching the door from the cannula to open the elongated slot and passing the elongated slot over the intramedullary nail.

In one embodiment, the at least one tissue dilator preferably comprises a plurality of cannulated tissue dilators having progressively larger diameters. A first one of the cannulated tissue dilators desirably has an inner diameter that is slightly larger than an outer diameter of the guide wire and a second one of the cannulated tissue dilators desirably has an inner diameter that is slightly larger than an outer diameter of the first cannulated tissue dilator. In one embodiment, each of the tissue dilators preferably tapers outwardly between distal and proximal ends thereof so that outer diameters of the tissue dilators at the distal ends thereof are smaller than outer diameters of the tissue dilators at the proximal ends thereof.

In one embodiment, the system preferably includes a cannulated illuminating device insertable into the cannula for illuminating the cortex of the bone. In one embodiment, the illuminating device preferably includes an outer wall having a proximal end and a distal end, light emitting elements adapted to project light from the distal end of the outer wall, and an elongated opening in the outer wall of the illuminating device that extends from the proximal end to the distal end of the outer wall of the illuminating device. In one embodiment, a cross-section of the outer wall of the cannulated illuminating device preferably defines a C shape. The cannulated illuminating device is preferably insertable into the cannula so that the elongated opening in the outer wall of the cannulated illuminating device is substantially aligned with the elongated slot in the outer wall of the cannula.

In one embodiment, a system for percutaneously placing intramedullary nails includes a guide wire for forming a percutaneous opening in tissue and engaging a cortex of a bone, at least one tissue dilator advanceable over the guide wire for widening the tissue opening, and a cannula advanceable over the at least one tissue dilator for further widening the tissue opening and providing access to the cortex of the bone. The cannula preferably has an outer wall extending between proximal and distal ends thereof, the outer wall including an elongated slot extending from the proximal end to the distal end of the cannula and a door for closing the elongated slot, whereby the door is selectively detachable from the outer wall for opening the elongated slot.

In one embodiment, a system for percutaneously placing intramedullary nails desirably includes a cannula insertable into a percutaneous tissue opening for providing access to a cortex of a bone, the cannula having an outer wall extending between proximal and distal ends thereof, the outer wall having a smaller portion including a door that extends between the proximal and distal ends of the cannula and a larger portion that bounds the door, whereby the door is selectively detachable from the larger portion of the outer wall for opening an elongated slot in the outer wall that extends from the proximal end to the distal end of the cannula. An intramedullary nail is preferably insertable into the cannula for engaging the cortex of the bone, whereby while the intramedullary nail engages the bone, the cannula is moveable away from the intramedullary nail by detaching the door from the cannula and passing the elongated slot of the cannula over the intramedullary nail.

These and other preferred embodiments of the present invention will be described in more detail below.

### BRIEF DESCRIPTION OF THE DRAWING

FIG. 1 shows a guide wire for intramedullary nail placement system, in accordance with one embodiment of the present invention.

FIG. 2 shows a threaded guide wire for an intramedullary nail placement system, in accordance with one embodiment of the present invention.

FIG. 3 shows an elongated guide wire for an intramedullary nail placement system, in accordance with one embodiment of the present invention.

FIGS. 4A-4D show a set of tissue dilators for an intramedullary nail placement system, in accordance with one embodiment of the present invention.

FIGS. 5A-5C show respective side, top plan, and cross-sectional views of a cannula for an intramedullary nail placement system, in accordance with one embodiment of the present invention.

FIGS. 6A-6C show respective side, top plan, and cross-sectional views of a cannula for an intramedullary nail placement system, in accordance with one embodiment of the present invention.

FIGS. 7A-7C show a method of removing a sliding door from the cannula shown in FIGS. 5A-5C and 6A-6C, in accordance with one embodiment of the present invention.

FIGS. 8A-8C show respective side, top plan, and cross-sectional views of a cannula for an intramedullary nail placement system, in accordance with one embodiment of the present invention.

FIGS. 9A and 9B show respective side and cross-sectional views of a light generating device, in accordance with one embodiment of the present invention.

FIG. 10A shows the light generating device of FIG. 9B inserted into the cannula of FIG. 5B, in accordance with one embodiment of the present invention.

FIGS. 11A-11O show a method of placing an intramedullary nail in an intramedullary canal of a long bone, in accordance with one embodiment of the present invention.

FIGS. 11L-1 through 11N-1 show cross-sectional views of the method steps shown in respective FIGS. 11L-11N, in accordance with one embodiment of the present invention.

FIG. 12 shows a cannula for an intramedullary nail placement system, in accordance with one embodiment of the present invention.

FIG. 13 shows a cannula for an intramedullary nail placement system, in accordance with another embodiment of the present invention.

FIGS. 14A and 14B show a cannula having a detachable handle, in accordance with one embodiment of the present invention.

### DETAILED DESCRIPTION

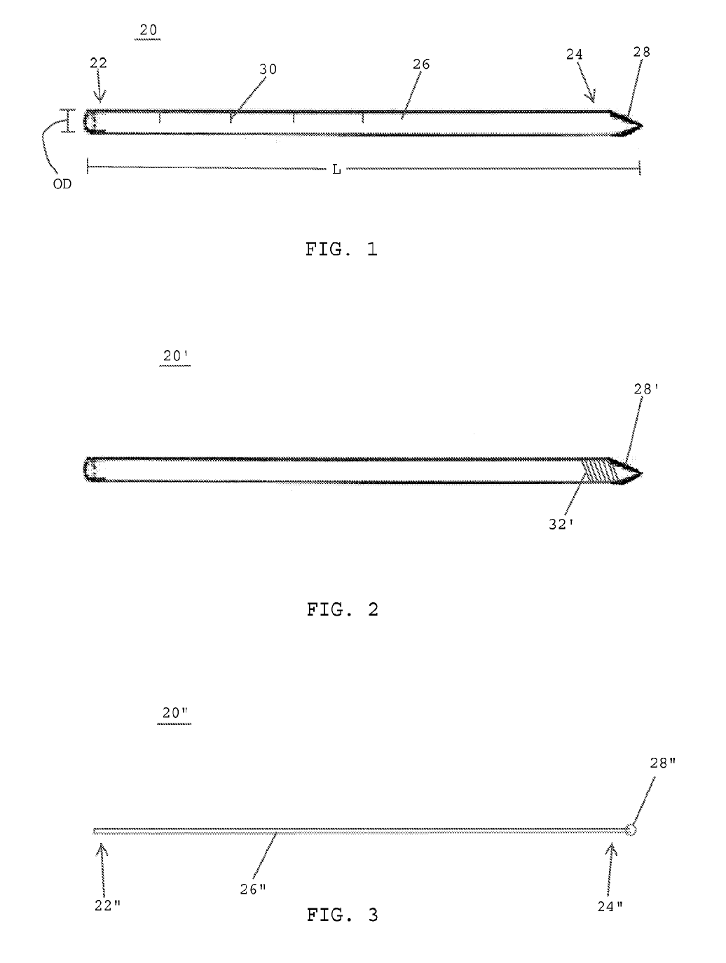

Referring to FIG. 1, in one embodiment, an intramedullary nail placement system includes a guide wire 20 having a proximal end 22, a distal end 24, and an elongated shaft 26 that desirably extends between the proximal and distal ends. The elongated shaft 26 of the guide wire 20 is preferably smooth. The distal end 24 of the guide wire 20 desirably has a pointed tip 28 for passing through skin, soft tissue and bone. The pointed tip 28 may also function to hold the guide wire 20 in place after the distal end 24 has been inserted into bone.

In one embodiment, the guide wire 20 is preferably made of stainless steel. In one embodiment, the guide wire is made of a durable, biocompatible material. In one embodiment, the guide wire 20 has an outer diameter OD of approximately 3.0 mm and a length L of between about 32-150 cm. In one embodiment, the guide wire 20 preferably includes visual indicia 30 provided along the outer surface of the elongated shaft 26 to provide an indication of the length of the guide wire inserted through soft tissue and into bone.

In one embodiment, the sharpened distal end 24 of the guide wire 20 is preferably adapted to be placed percutaneously through the skin epidermis and the soft tissue until it engages the bone cortex of the long bone at a desired starting position. In one embodiment, the distal end 24 of the guide wire 20 is preferably advanced through the bone cortex at the desired starting position and into the medullary canal of the long bone without requiring the use of a scalpel for sharp dissection of the skin and soft tissues.

The visual indicia 30 provided on the outer surface of the elongated shaft 26 may be observed so that the length of the soft tissue that is traversed by the guide wire 20 from the skin to the bone cortex may be noted and recorded. As will be described in more detail below, this measurement is preferably used to determine the required length for soft tissue dilators and cannulated cylinders that are used in subsequent steps of a medullary nail placement procedure.

In one embodiment, the location in the long bone of the patient where the guide wire 20 must engage and pass across the bone cortex is desirably selected using fluoroscopic imaging and/or digital palpation. In one preferred embodiment, fluoroscopy is utilized to indicate whether the guide wire 20 is situated in an acceptable position in the long bone's cortex and intramedullary canal.

Referring to FIG. 2, in one embodiment, a guide wire 20′ has all of the features described above for the guide wire 20 shown in FIG. 1, and includes threads 32′ provided adjacent a distal tip 28′. As the guide wire 20′ is advanced through bone, the threads 32′ preferably hold the guide wire 20′ to the bone by providing an enhanced gripping surface for engaging the bone. In one embodiment, the smooth guide wire 20 of FIG. 1 is used when initially passing a guide wire through skin and soft tissue. After an opening exposing the bone has been formed, the threaded guide wire 20′ may be used to better secure the guide wire to the bone.

Referring to FIG. 3, in one embodiment, an intramedullary nail system preferably includes an elongated, flexible guide wire 20″ having a proximal end 22″, a distal end 24″, and an elongated, flexible shaft 26″ that is adapted to advance through an intramedullary canal. The distal end 24″ of the elongated guide wire 20″ preferably includes a dull of ball tip 28″ that minimizes the likelihood of the distal end 24″ piercing through bone or tissue. The elongated guide wire 20″ preferably has a length that enables it to span the length of an intramedullary canal of a long bone. As noted above, the shaft 26″ is desirably flexible so that the shaft can flex and bend to conform to the intramedullary canal of the long bone as the guide wire 20″ advances through the canal.

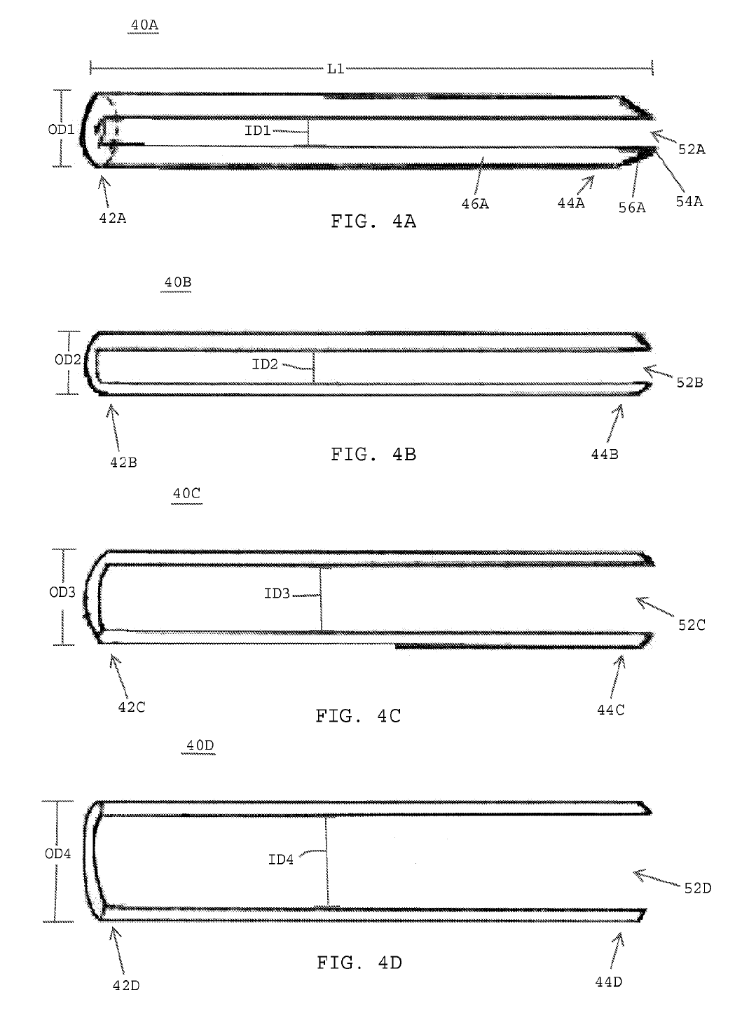

Referring to FIGS. 4A-4D, in one embodiment, an intramedullary nail placement system desirably includes a series of progressively larger tissue dilators that are adapted to slide over one another for increasing the size of an initial percutaneous opening formed by one of the guide wires shown in FIGS. 1 and 2. In one embodiment, a first tissue dilator is preferably adapted to slide over a guide wire, a second tissue dilator is adapted to slide the first tissue dilator, a third tissue dilator is adapted to slide over the second tissue dilator, etc., until an adequately sized opening is formed in soft tissue.

Referring to FIG. 4A, in one embodiment, a first tissue dilator 40A includes a proximal end 42A, a distal end 44A, and an outer wall 46A extending between the proximal and distal ends thereof. The outer wall 46A preferably includes an outer surface 48A defining an outer diameter OD₁ and an inner surface 50A defining an inner diameter ID₁. The inner surface 50A of the outer wall 46A desirably defines an elongated opening 52A that extends from the distal end 44A toward the proximal end 42A of the first tissue dilator 40A. The distal end 44A of the outer wall 46A preferably includes a sharpened point 54A that has an outwardly sloping surface 56A that slopes outwardly between the distal and proximal ends of the tissue dilator. In one embodiment, the length L₁ of the first tissue dilator 40A is preferably about 10 cm longer than the recorded distance of the soft tissue traversed by the guide wire 20.

The inner diameter ID₁ of the elongated opening 52A preferably closely matches and conforms to the outer diameter OD of the guide wire 20 shown in FIG. 1. In one embodiment, the first soft tissue dilator 40A preferably has an outer diameter OD₁ of approximately 6.0 mm and an inner diameter ID₁ of about 3.1 mm. As noted above, the opening 52A at the distal end 44A of the first tissue dilator 40A is preferably adapted for being guided over the outer surface of the shaft 26 of the guide wire 20 (FIG. 1). In one embodiment, the first tissue dilator 40A is preferably guided over the first guide wire 20 so that the distal end 44A of the first dilator passes through the skin and the soft tissue until the sharpened point 54A of the first dilator engages the bone cortex at the entry site of the guide wire into the long bone.

Referring to FIGS. 4B-4D, in one embodiment, an intramedullary nail placement system preferably includes a set of additional soft tissue dilators 40B, 40C and 40D of gradually increasing inner and outer diameters. The sequentially larger dilators 40B, 40C, 40D have generally the same structure as described above for the first tissue dilator 40. In one embodiment, the second soft tissue dilator 40B is adapted to slide over and closely conform to the outer surface of the first soft tissue dilator 40A, the third soft tissue dilator 40C is adapted to slide over and closely conform to the outer surface of the second soft tissue dilator 40B, and the fourth soft tissue dilator 40D is adapted to slide over and conform to the outer surface of the third soft tissue dilator 40C. Referring to FIGS. 4A and 4B, in one embodiment, the inner diameter ID₂ of the second tissue dilator 40B closely conforms to and slides over the outer diameter OD₁ of the first tissue dilator 40A. Referring to FIGS. 4B and 4C, in one embodiment, the inner diameter ID₃ of the third tissue dilator 40C closely conforms to and slides over the outer diameter OD₂ of the second tissue dilator 40B. Referring to FIGS. 4C and 4D, in one embodiment, the inner diameter ID₄ of the fourth tissue dilator 40D closely conforms to and slides over the outer diameter OD₃ of the third tissue dilator 40C.

Although a set of four soft tissue dilators is shown in FIGS. 4A-4D, in other embodiments, sets having more than four soft tissue dilators may be provided. In one embodiment, a sufficient number of soft tissue dilators are preferably advanced over the prior soft tissue dilator until a dilator of adequate diameter necessary to accommodate the appropriate hardware components of the intramedullary nail placement system is obtained.

In one embodiment, the inner and outer diameters of each of the subsequent soft tissue dilators preferably increases by increments of approximately 3.0 mm to a maximum inner diameter of 18.1 mm and a maximum outer diameter of 21 mm. In one embodiment, the tissue dilators of a set have lengths of between about 15-60 cm. The respective lengths of the tissue dilators in a set may increase in increments of about 15 cm.

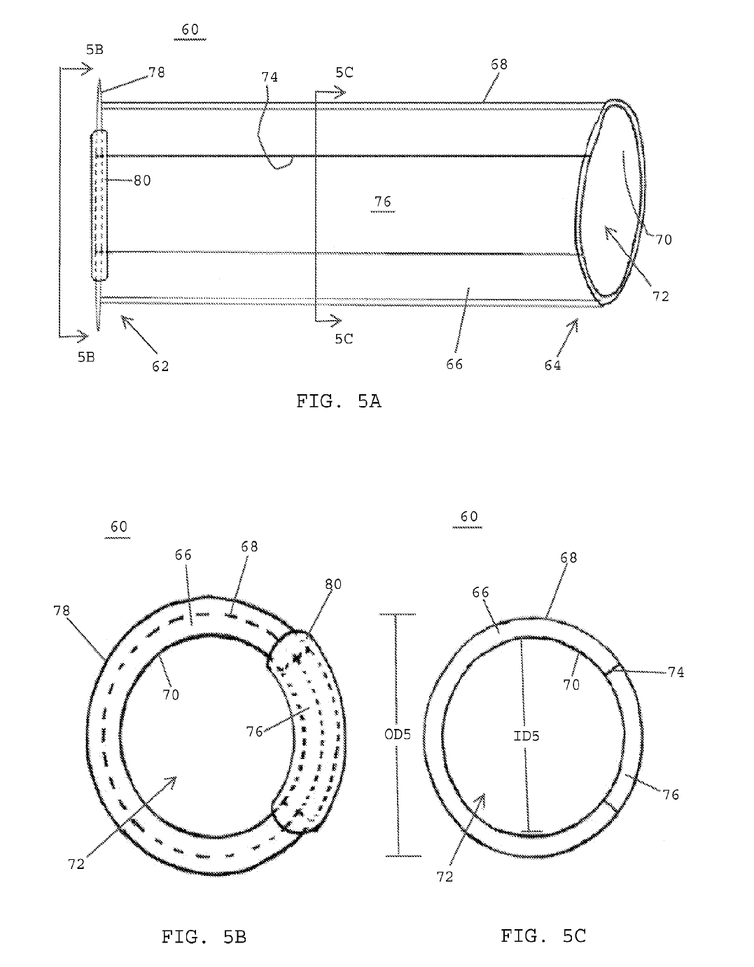

Referring to FIG. 5A-5C, in one embodiment, an intramedullary nail placement system preferably includes a cannula 60 adapted to pass through soft tissue for providing access to bone at a surgical site. In one preferred embodiment, the cannula 60 is adapted to slide over and closely conform to the outer surface of a tissue dilator. The cannula 60 desirably has a proximal end 62, a distal end 64, and an outer wall 66 that extends between the proximal and distal ends. The distal end 64 of the cannula 60 may have various shapes including a flat surface, a recessed center, and an oblique shape, as will be described in more detail below. The cannula 60 may also be provided with an optional handle attachment having a threaded screw head of 0.8 mm in depth that preferably matches the threaded recessed portion at the proximal end of the cannula.

In one embodiment, the outer wall preferably has a cylindrical shaped outer surface 68 defining an outer diameter OD₅, and a cylindrical shaped inner surface 70 defining an inner diameter ID₅. In one embodiment, the inner surface 70 defines a cylindrical shaped opening 72 that extends between the proximal and distal ends of the cannula. In one embodiment, the outer surface 60 of the outer 68 has an outer diameter OD₅ of approximately 22.0 mm and the inner surface 70 of the outer wall 66 has an inner diameter ID₅ of approximately 21.1 mm. In one embodiment, the inner diameter ID₅ preferably closely matches and conforms to the outer diameter OD₄ of the fourth tissue dilator 40D (FIG. 4D). The cannula 60 preferably has an inner diameter that is large enough to accommodate the outer diameter of the intramedullary nail component being passed therethrough.

In one embodiment, the cannula 60 preferably has a length that is longer than the length of the soft tissue traversed by the first guide wire 20 (FIG. 1). In one embodiment, the cannula 60 preferably has a length that is less than the length of the respective soft tissue dilators 40A-40D (FIGS. 4A-4D). In one embodiment, the cannula 60 preferably has a length of about 10-55 cm. In one embodiment, a plurality of cannulas having different lengths may be provided whereby the lengths of the respective cannulas increase in increments of about 15 cm.

In one embodiment, the cannula 60 preferably includes an elongated slot 74 that extends from the proximal end 62 to the distal end 64 of the cannula. The elongated slot 74 is preferably covered by a sliding door 76 that may be slid distally for completely covering the elongated slot 74, and slid proximally for detaching the sliding door 76 from the cannula 60 and completely opening the elongated slot 74. When the sliding door 76 is closed, the sliding door forms parts of the outer wall 66 of the cannula. When the sliding door 76 is removed, the elongated slot 74 defines a rectangular shaped opening that extends the length of the cannula, thereby giving the cannula a C-shaped appearance when viewed in cross-section.

In one embodiment, the elongated slot 74 and the sliding door may have opposing projections and grooves for guiding sliding movement of the door 76 along the longitudinal axis of the cannula 60. The tongue and groove structure also preferably enhances the structural integrity of the cannula when the sliding door 76 is in the closed position shown in FIG. 5A. The elongated slot also preferably includes a stop located adjacent the distal end 64 of the cannula 60 for preventing further distal movement of the door 76 when it reaches the position shown in FIG. 5A.

In one embodiment, the proximal end 62 of the cannula 60 preferably includes an annular rim 78 that has a slightly larger outer diameter than the outer diameter OD₅ of the outer surface 68 of the outer wall 66. The cannula 60 preferably includes a slidable locking ring 80 that is adapted to slide over the outer surface of the annular rim 78.

Referring to FIGS. 5A and 5B, in one embodiment, the locking ring 80 may be placed in a locked position whereby it covers the proximal end of the sliding gate 76. When the locking ring 80 is in the locked position (FIG. 5B), the sliding door 76 may not move toward the proximal end 62 of the cannula 60. As a result, the sliding door 76 may not be detached from the cannula 60 for opening the elongated slot 74.

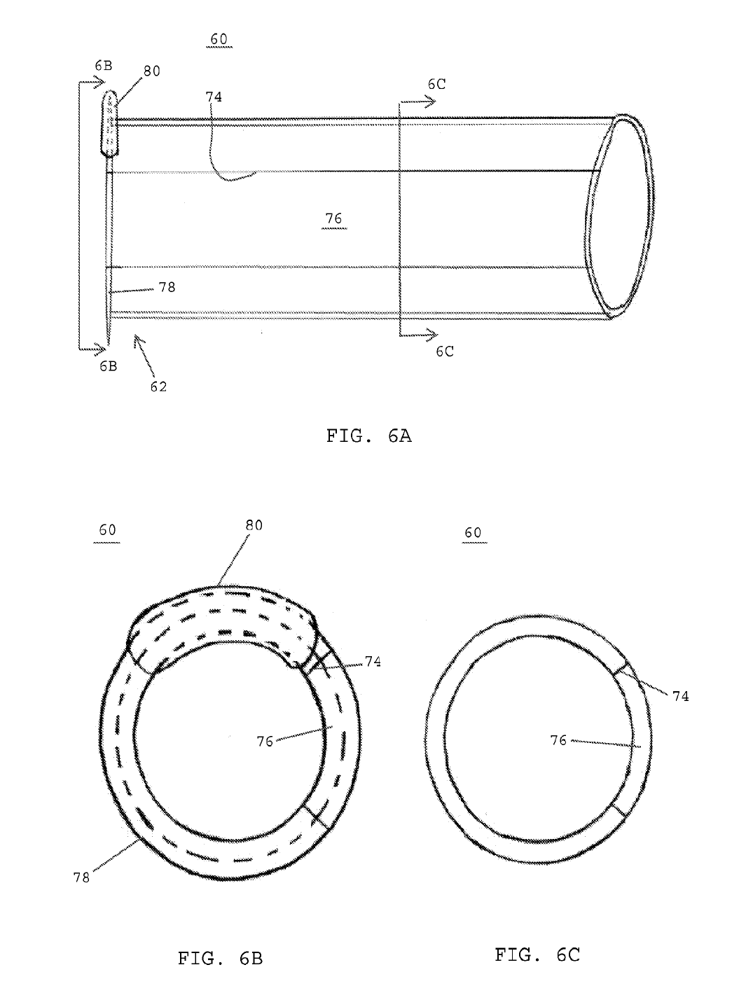

Referring to FIGS. 6A-6C, in one embodiment, the locking ring 80 is preferably moveable over the outer surface of the rim 78 until it reaches an unlocked position in which it does not cover the proximal end of the sliding door 76. With the locking ring 80 in the unlocked position, the sliding door 76 is able to move toward the proximal end 62 of the cannula for detaching the door from the cannula 60 so as to open the elongated slot 74.

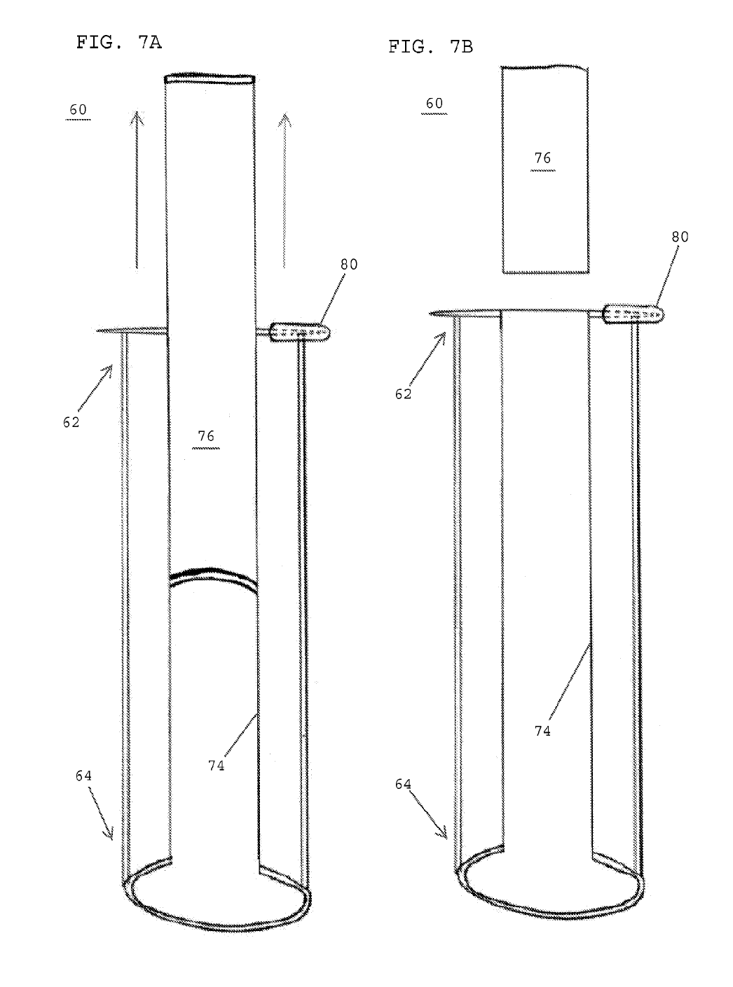

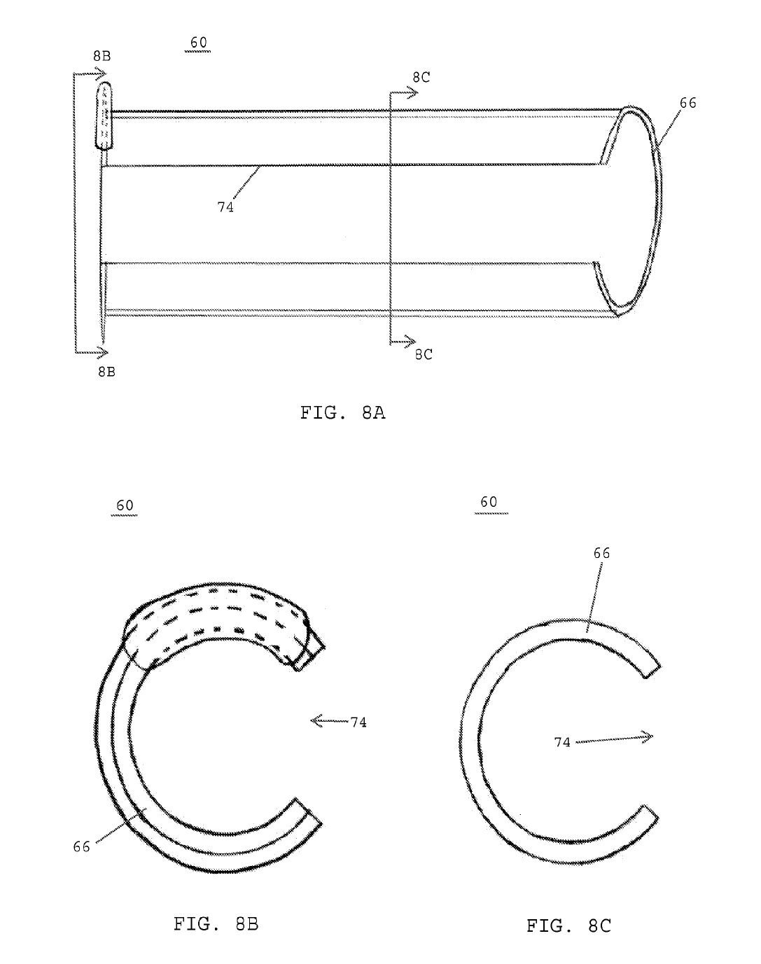

Referring to FIGS. 7A and 7B, in one embodiment, with the locking ring 80 in the unlocked position, the sliding door 76 is free to slide toward the proximal end 62 of the cannula 60 for opening the elongate slot 74 that extends from the proximal end 62 to the distal end 64 of the cannula 60. FIG. 7B shows the cannula 60 after the sliding door 76 has been completely detached therefrom to completely open the elongated slot 74. As shown in FIG. 7B, the when the sliding door 76 is completely removed, the elongated slot 74 defines a rectangular shaped opening that extends through the outer wall 66 and along the entire length of the cannula 60.

FIGS. 8A-8C show the cannula 60 after the sliding door has been removed. The outer wall 66 of the cannula 60 defines the elongated slot 74, which extends the length of the cannula. As shown in FIGS. 8B and 8C, with the sliding door detached, the outer wall 66 of the cannula 60 defines a C-shaped structure having an opening that extends along one side of the cannula. Although the present invention is not limited by any particular theory of operation, it is believed that providing a cannula having an elongated slot extending along the entire length thereof enables intramedullary nails to be placed percutaneously because the cannula can be removed by passing the nail through the elongated slot of the cannula. This cannot be accomplished using prior art cannulas that having 360 degree walls at the ends of the cannula.

Referring to FIGS. 6A and 6B, in one embodiment, an intramedullary nail placement system preferably includes an illuminating device 82 that is preferably adapted to slide inside the cannula 60 (FIGS. 5A-5C) for providing light and visibility at the distal end of the cannula, which is preferably at a surgical site. In one embodiment, the illuminating device 82 preferably has a proximal end 84, a distal end 86, and an outer wall 88 extending between the proximal and distal ends. The outer wall 88 preferably defines a cylindrical shaped outer surface 90 defining an outer diameter OD₆ and an inner surface 92 defining an inner diameter ID₆. In one embodiment, the illuminating device 82 desirably has an outer diameter OD₆ that is adapted to closely conform with the inner diameter ID₅ of the cannula 60.

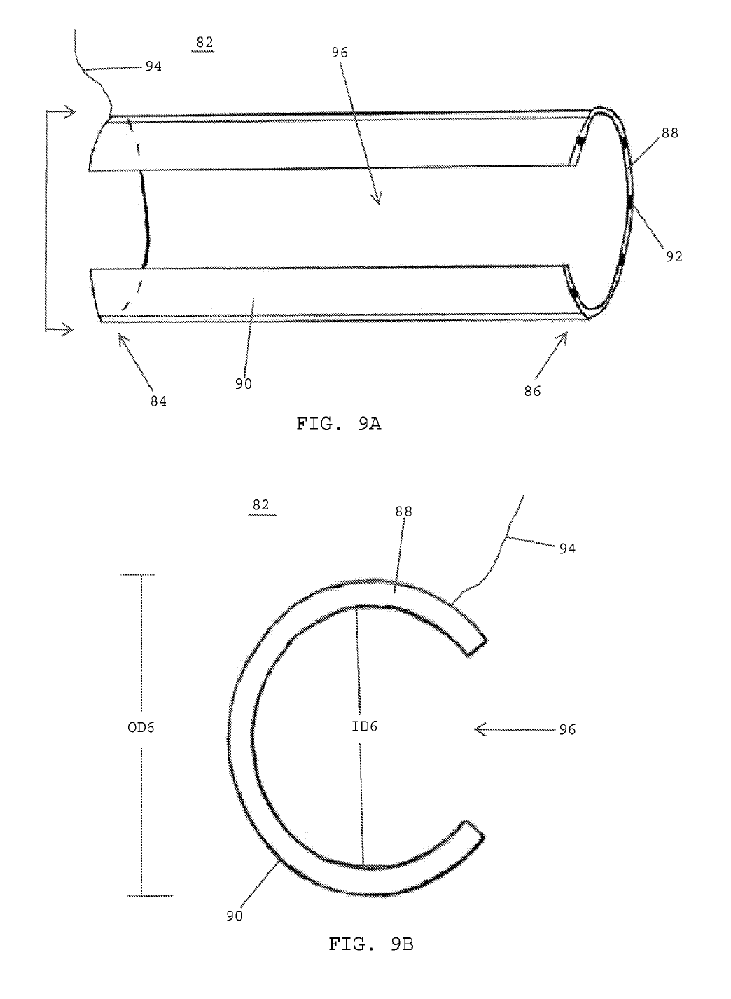

Referring to FIG. 9A, in one embodiment, the distal end 86 of the illuminating device 82 preferably includes light generating elements 92, such as light-emitting diodes (LEDs) or optical fibers, that are adapted to project light from the distal end 86 of the illuminating device. The projected light preferably illuminates a surgical site such as bone at a surgical site. The illuminating device 82 may include one or more conductive elements 94 for providing power and/or control signals to the light generating elements 92.

Referring to FIGS. 9A and 9B, in one embodiment, the outer wall 88 of the illuminating device 82 preferably has an elongated opening 96 extending between the proximal and distal ends 84, 86 thereof. The elongated opening 96 gives the illuminating device 82 a C-shaped appearance when viewed in cross-section (FIG. 9B).

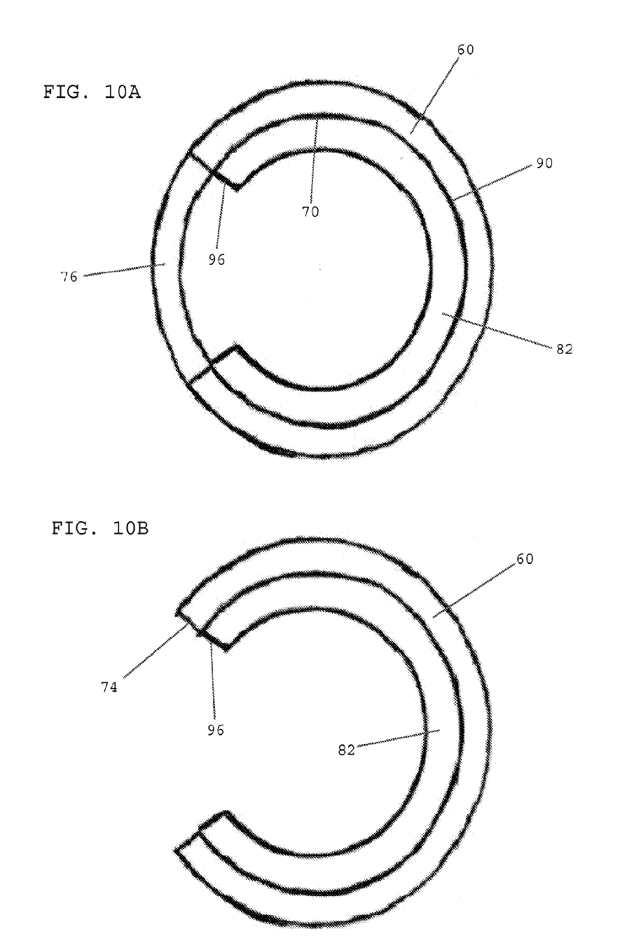

Referring to FIGS. 10A and 10B, in one embodiment, the illuminating device 82 is adapted to slide inside with cannula 60. In one embodiment, the outer surface 90 of the illuminating device 82 is adapted to closely conform with the inner surface 70 of the cannula 60. In one embodiment, the illuminating device and the cannula are joined together so that the elongated opening 96 in the illuminating device 80 is in alignment with the sliding door 76 of the cannula 60. FIG. 10A shows the illuminating device 82 and the cannula 60 when the door 76 covers the elongated slot 74 of the cannula 60. FIG. 10B shows the illuminating device 82 and the cannula 60 after the door has been removed to expose the elongated slot 74, which is preferably in alignment with the elongated opening 96 of the illuminating device 82. As will be described in more detail below, the respective elongated openings 74, 96 preferably enable the illuminating device and the cannula to slide over an intramedullary nail and/or an alignment device for an intramedullary nail when it is desirable to remove the illuminating device and the cannula from a surgical site.

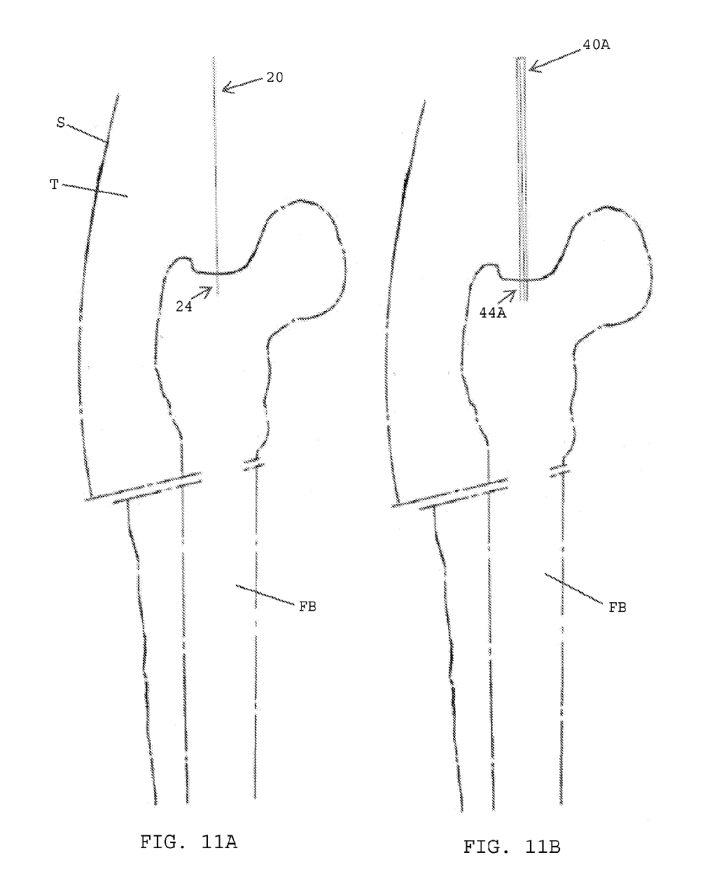

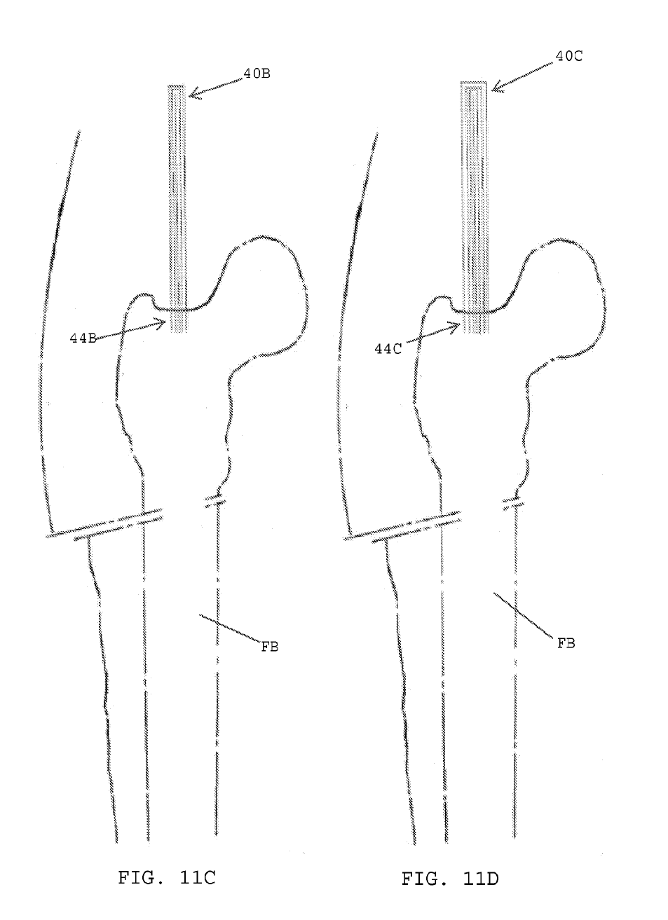

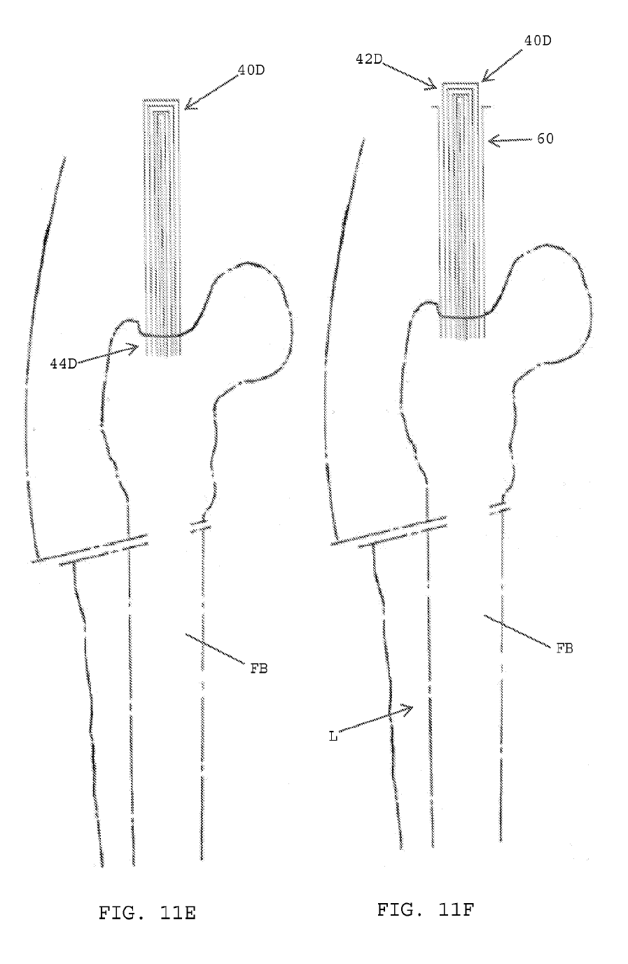

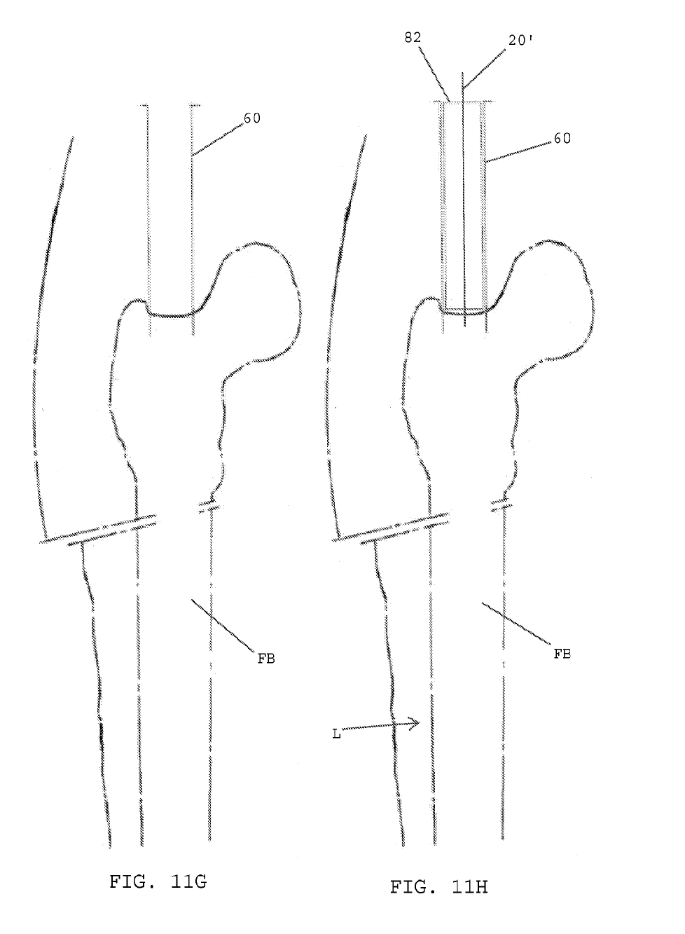

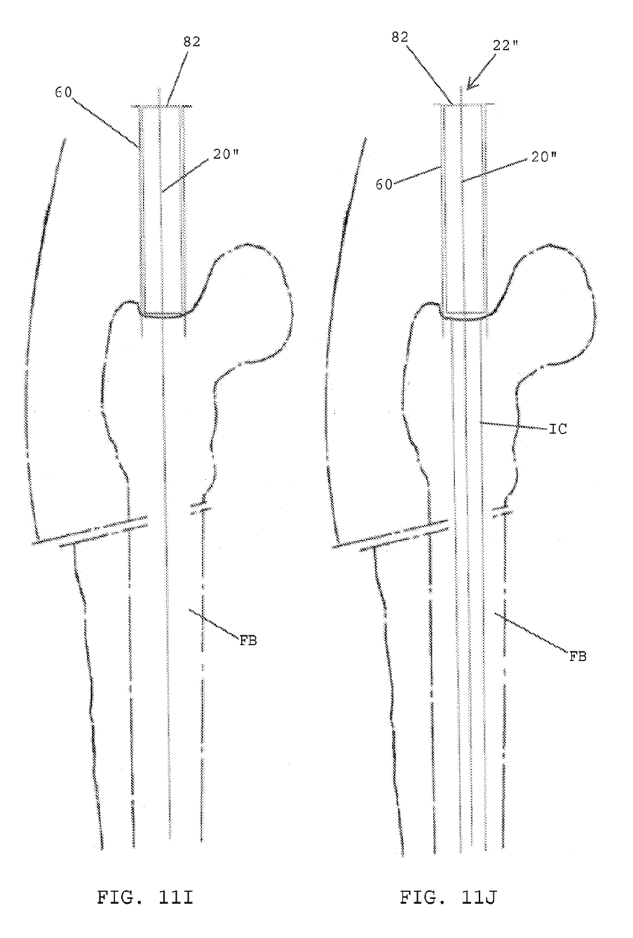

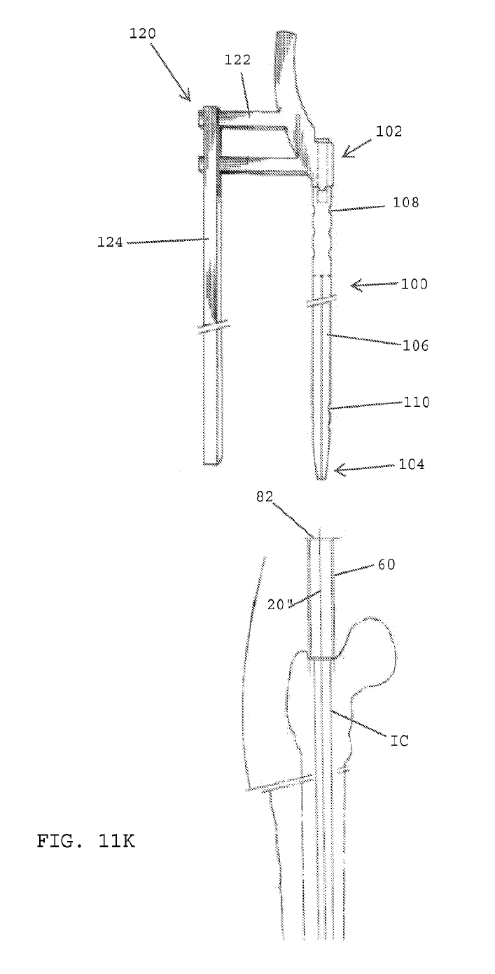

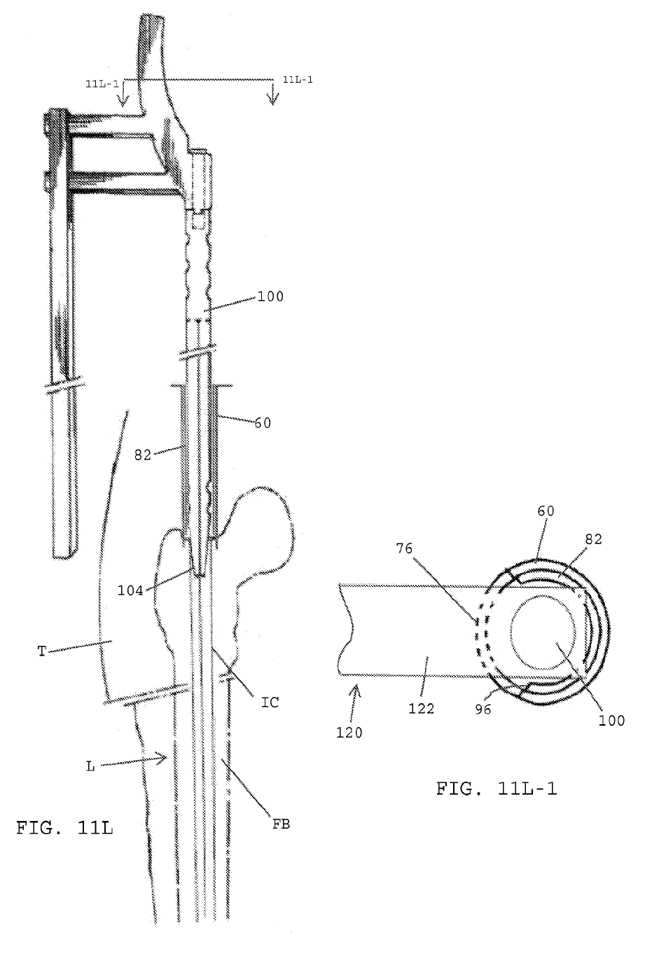

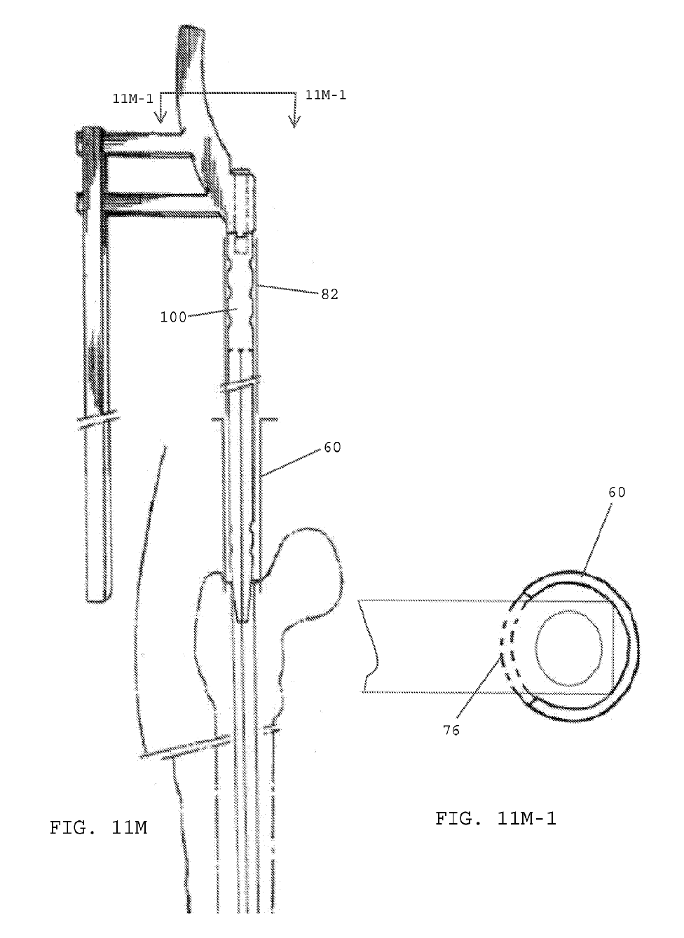

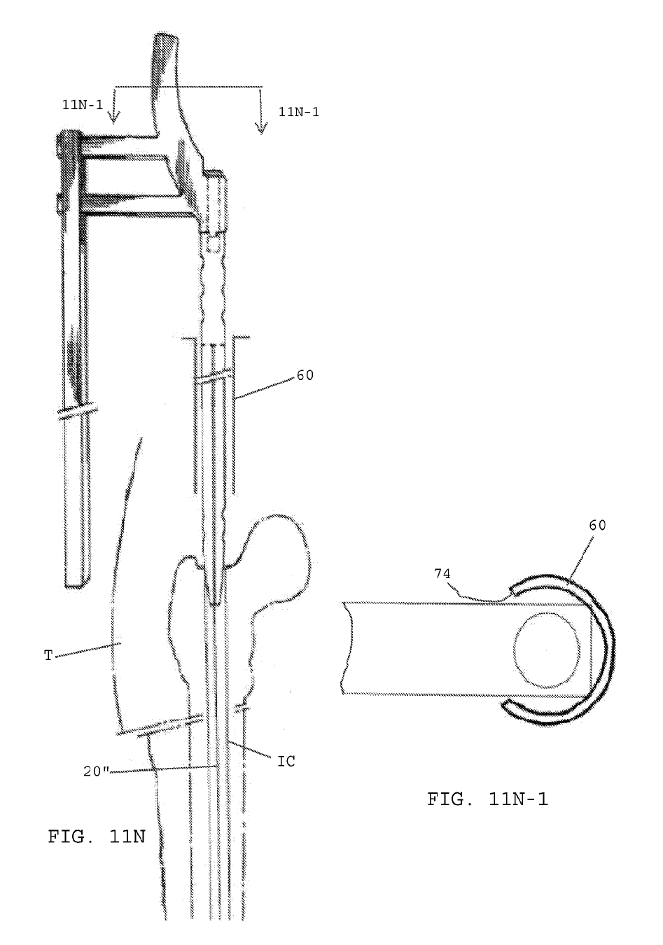

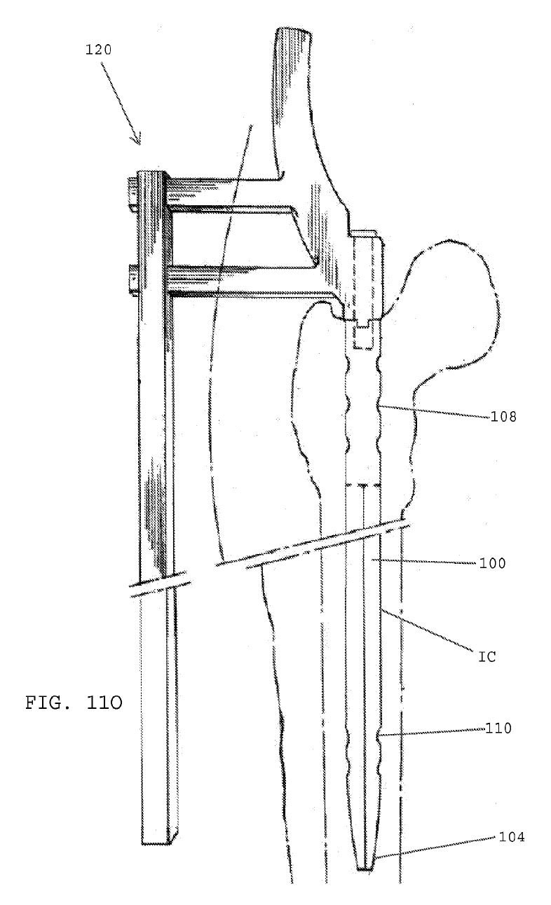

In one embodiment, the intramedullary nail placement system described herein may be utilized for placing intramedullary nails within long bones using a percutaneous, minimally invasive procedure, and without requiring the sharp dissection of soft tissue. Referring to FIGS. 1 and 11A, in one embodiment, the pointed tip 28 at the distal end 24 of a first guide wire 20 may be advanced through the skin S and soft tissue T of a patient until the distal end is anchored in cortical bone located at an upper end of a femur bone FB. The distal end 24 of the guide wire is preferably aligned with the intramedullary canal of the femur bone FB. The visual indicia 30 provided on the shaft 26 of the guide wire 20 are observed to determine the length of the guide wire that passed through the soft tissue. The surgeon preferably uses the measured length for the guide wire to select appropriately sized tissue dilators for a later stage of the procedure.

Referring to FIGS. 4A and 11B, the opening 52A at the distal end 44A of the first tissue dilator 40A is preferably aligned with proximal end of the guide wire 20 previously anchored in the upper end of the femur bone FB. The distal end 44A of the first tissue dilator 40A preferably slides over the outer surface of the guide wire 20 and toward the femur bone FB until the distal end 44A is anchored in the bone. The first tissue dilator 40A preferably widens the percutaneous opening made by the first guide wire.

Referring to FIGS. 4B and 11C, the opening 52B at the distal end 44B of the second tissue dilator 40B is preferably aligned with proximal end 42A of the first tissue dilator 40A previously anchored in the upper end of the femur bone FB. The distal end 44B of the second tissue dilator 40B preferably slides over the outer surface of the first tissue dilator 40A and toward the femur bone FB until the distal end 44B is anchored in the bone. The second tissue dilator 40B preferably widens the percutaneous opening made by the first tissue dilator 40A.

Referring to FIGS. 4C and 11D, the opening 52C at the distal end 44C of the third tissue dilator 40C is preferably aligned with proximal end 42B of the second tissue dilator 40B previously anchored in the upper end of the femur bone FB. The distal end 44C of the third tissue dilator 40C preferably slides over the outer surface of the second tissue dilator 40B and toward the femur bone FB until the distal end 44C is anchored in the bone. The third tissue dilator 40C preferably widens the percutaneous opening made by the second tissue dilator 40B.

Referring to FIGS. 4D and 11E, the opening 52D at the distal end 44D of the fourth tissue dilator 40D is preferably aligned with proximal end 42C of the third tissue dilator 40C previously anchored in the upper end of the femur bone FB. The distal end 44D of the fourth tissue dilator 40D preferably slides over the outer surface of the third tissue dilator 40C and toward the femur bone FB until the distal end 44D is anchored in the bone. The fourth tissue dilator 40D preferably widens the percutaneous opening made by the third tissue dilator 40C.

In one embodiment, additional tissue dilators may be used for widening the percutaneous opening. The number of dilators used may vary and will desirably depend upon the size of the percutaneous opening required for performing a successful surgical procedure.

Referring to FIGS. 5A and 11F, the circular opening 72 at the distal end 64 of the cannula 60 is desirably aligned with the proximal end 42D of the fourth tissue dilator 40D (FIG. 4D) previously anchored in the upper end of the femur bone FB. The distal end 64 of the cannula 60 preferably slides over the outer surface of the fourth tissue dilator 40D and toward the femur bone FB until the distal end 64 of the cannula is anchored in the bone. The cannula 60 preferably widens the percutaneous opening made by the fourth tissue dilator 40D. When the cannula 60 slides over the fourth tissue dilator, the sliding door on the cannula is preferably closed and facing toward the lateral side L of the femur bone FB.

Referring to FIG. 11G, after the cannula 60 is anchored to the femur bone FB, the tissue dilators are preferably removed from the surgical site, leaving the cannula 60 in place over a target location on the femur bone. Referring to FIGS. 1, 2 and 11H, in one embodiment, the first smooth wire guide 20 is removed and replaced with the second threaded wire guide 20′. Referring to FIGS. 9A and 11H, in one embodiment, the distal end 86 of the illuminating device 82 is preferably aligned over the cannula 60 and slid within the cannula toward the bone at the surgical site. The light emitting elements 92 at the distal end 86 of the illuminating element 82 preferably face toward the bone for illuminating the surgical site. The elongated opening 96 formed in the sidewall 88 of the illuminating device is preferably aligned with the sliding door 76 (FIG. 5A) of the cannula 60. In other words, the elongated opening 96 faces toward the lateral side L of the femur bone FB.

Referring to FIGS. 2, 3 and 11I, in one embodiment, the second threaded guide wire 20′ is detached from the femur bone FB and the distal end 24″ of the flexible, elongated guide wire 20″ is passed through the central openings in the cannula 60 and the illuminating device 82. The distal end of the elongated guide 20″ is preferably advanced through the intramedullary canal of the femur bone FB. The dull ball 28″ at the distal end 24″ of the elongated guide wire 20″ desirably minimizes the likelihood that the distal end of the elongated guide will pierce through the femur bone FB. When the flexible, elongated wire guide 20″ has reached the distal end of the intramedullary canal IC, fluoroscopy and/or X-ray may be used to confirm that the elongated wire guide is properly positioned with the intramedullary canal IC.

Referring to FIG. 11J, in one embodiment, after fluoroscopy and/or X-ray have confirmed that the elongated guide wire 20″ is properly positioned within the intramedullary canal IC, a cannulated reamer (not shown) may be used for reaming the intramedullary canal IC. In one embodiment, the cannulated reamer is preferably aligned with the proximal end 22″ of the flexible, elongated wire guide 20″. The cannulated reamer preferable slides over and is guided by the elongated guide wire 20″ as the reamer advances toward the distal end of the femur. In one embodiment, in order to provide more space and/or protect the illuminating device 82, the illuminating device may be removed from the cannula 60 before the reamer is positioned inside the cannula. In this embodiment, the illuminating device 82 may be placed back inside the cannula 60 after the reaming operation is completed.

Referring to FIG. 11K, in one embodiment, after the intramedullary canal IC has been reamed, an intramedullary nail 100 is preferably aligned over the reamed canal. In one embodiment, the intramedullary nail preferably has a proximal end 102, a distal end 104, and an elongated shaft 106 extending between the proximal and distal ends. The intramedullary nail 100 also preferably includes a first set of laterally extending openings 108 adjacent the proximal end 102 of the nail 100 and a second set of laterally extending openings 110 adjacent the distal end 104 of the nail 100. The laterally extending openings are preferably adapted to receive proximal and distal anchoring screws for preventing rotation or twisting of the nail 100 within the intramedullary canal IC. In one embodiment, the intramedullary nail 100 is desirably cannulated and has a central opening extending along the length thereof.

In one embodiment, the proximal end 102 of the intramedullary nail 100 is preferably coupled with an alignment device 120 having at least one horizontally extending member 122 and at least one vertically extending member 124. The vertically extending member 124 preferably includes laterally extending opening that may be used for aligning anchoring screws with the laterally extending openings 108, 110 of the intramedullary nail 100.

Referring to FIG. 11K, with the intramedullary nail 100 secured to the alignment device 120, the distal end 104 of the nail is desirably aligned with the respective circular openings at the upper ends of the cannula 60 and the illuminating device 82. During this stage of the procedure, the elongated guide wire 20″ may remain in the reamed intramedullary canal IC or it may be removed from the reamed canal.

Referring to FIG. 11L, in one embodiment, the distal end 104 of the intramedullary nail 100 is preferably advanced through the central openings of the cannula 60 and the illuminating device 82 and into a proximal section of the reamed intramedullary canal IC. As the distal end 104 of the nail 100 is advanced into the canal, the illuminating device 82 preferably illuminates the surgical site so that the surgeon may confirm that the nail is properly positioned with the canal.

Once the surgeon has confirmed that the distal end of the nail 100 is properly positioned within the intramedullary canal IC, the illuminating device 82 and the cannula 60 may be removed from the surgical site. Referring to FIG. 11L-1, at the stage shown in FIG. 11L, the sliding door 76 of the cannula 60 remains closed. In one embodiment, the elongated opening 96 of the illuminating element 82 and the sliding door 76 of the cannula 60 are in substantial alignment with one another and may face toward the lateral side L of the femur bone FB.

In one embodiment, the distal end 104 of the nail 100 may remain within the canal IC as the illuminating device 82 and the cannula 60 are removed, which is a major focus of the present application and which provides a significant advantage over prior art intramedullary nail placement techniques. The elongated opening 96 on the illuminating device 82 (FIG. 9A) enables the illuminating device to be slid over the shaft of the nail 100 and removed from the surgical site as the nail remains in contact with the alignment device 120 and the intramedullary canal IC. The capability of removing the sliding door 76 to provide the elongated slot 74 in the cannula enables the cannula 60 to be slid over the shaft of the nail 100 as the nail remains in contact with the alignment device 120 and the intramedullary canal IC.

Referring to FIGS. 11M and 11M-1, in one embodiment, the illuminating device 82 is retracted from the inside if the cannula and the elongated opening of the illuminating device is passed over the intramedullary nail 100. The cannula 60 remains within the tissue T and sliding door 76 remains closed so that the tissue does not creep into the cannula.

Referring to FIGS. 11N and 11N-1, the cannula may be retracted from the surgical site and pulled from the tissue T, the sliding door 76 may then be removed to expose the elongated slot 74 of the cannula 60. The elongated slot 74 may be passed over the intramedullary nail 100 and removed from the surgical site. The flexible, elongated wire guide 20″ may be retracted from the intramedullary canal IC.

Referring to FIG. 11O, the intramedullary nail 100 may then be driven toward the distal end of the intramedullary canal IC until the nail 100 is fully seated in the canal. The alignment device 120 may then be used for aligning anchoring screws with the sets of lateral openings 108, 110 in the nail.

Referring to FIG. 12, in one embodiment, a cannula 160 for an intramedullary nail placement system includes a proximal end 162 and a distal end 164. The distal end 164 preferably includes a recess 165 formed in distal face that is adapted to conform to the contour of an opposing bone surface. The exact location of the recess 165 at the distal end face may be changed to accommodate various bones having different contours.

Referring to FIG. 13, in one embodiment, a cannula 260 for an intramedullary nail placement system has a proximal end 262 and a distal end 264 remote therefrom. The distal face of the cannula 260 preferably defines an oblique circumferential open face 265 that is preferably adapted to conform to the contour of an opposing bone surface.

Although FIGS. 12 and 13 show cannula embodiments having specific geometric shapes, it is contemplated that other cannulas may have distal end faces with different geometric shapes to accommodate opposing bone surfaces.

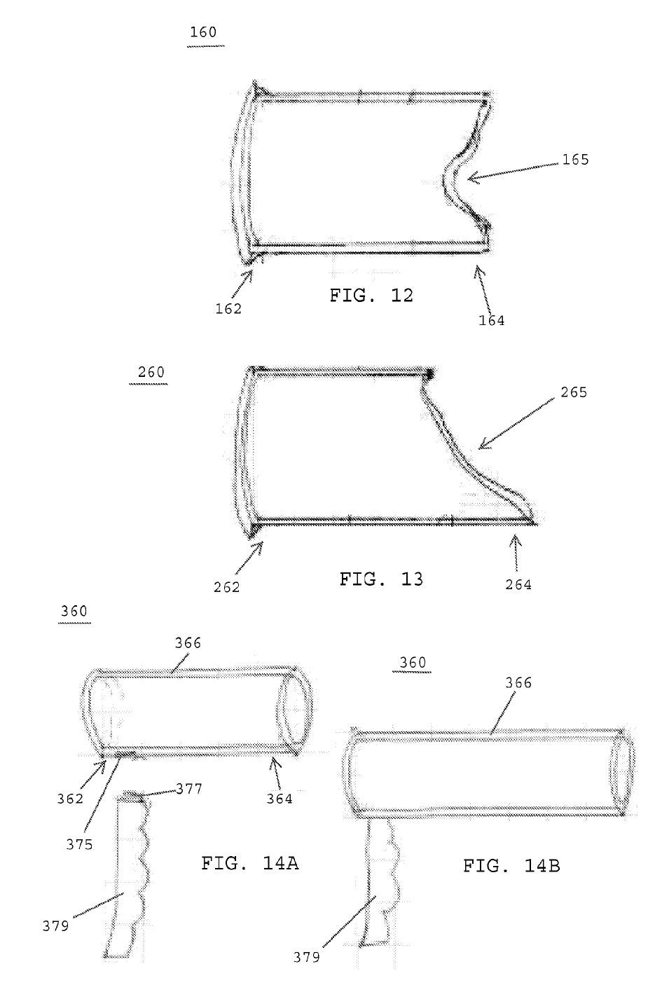

Referring to FIG. 14A, in one embodiment, a cannula 360 for an intramedullary nail placement system includes a proximal end 362 and a distal end 364 remote therefrom. The outer wall 366 of the cannula 360 desirably includes a threaded recess 375 adapted to receive a threaded projection 377 on a handle 379. The handle 379 may be de-coupled from the cannula 360 for performing certain steps of a surgical procedure. If desired, the handle 379 may be secured to the outer wall 366 of the cannula 360, as shown in FIG. 14B. Although the present invention is not limited by any particular theory of operation, it is believed that the detachable handle 379 may provide additional leverage for a surgeon during a surgical procedure.

In one embodiment, a second guide wire having an outer diameter of approximately 3.0 mm and a series of surrounding soft tissue dilators are preferably loaded into a cannula having no removable sliding sidewall or door. The cannula desirably fits within the radial bore on an external targeting device for placing lag screws that will cross the fracture site after engaging the radial bore in the intramedullary nail. In one embodiment, the cannula with the soft tissue dilators and the sharpened guide wire are preferably advanced into the corresponding radial bore in the external targeting device. The sharpened distal end of the guide wire is preferably advanced through the skin and the soft tissues until it engages the near cortex of the long bone. A fluoroscopic image is preferably taken to confirm an acceptable position of the guide wire where it engages the bone cortex. The length of the soft tissue traversed by the guide wire is desirably noted and recorded. The length of the soft tissue dilators and the length of the cannula are desirably selected so that they are able to span the soft tissue gap using the methods as outlined herein.

In one embodiment, after placement of the guide wire in the bone cortex, a soft tissue dilator having an inner diameter of 3.1 mm and an outer diameter of 6.0 mm is placed over the guide wire and a cannula with an outer diameter of 7.0 mm and an inner diameter of 6.1 mm having no handle attachment is placed over the tissue dilator having the outer diameter of 6.0 mm. In one embodiment, the cannula is preferably placed in the radial bore in the external targeting device and through the skin and soft tissue up to the point of engaging the outer bone cortex. The soft tissue dilators and the steel guide wire are preferably removed. The 7.0 mm outer diameter cannula is desirably left in place engaging the outer bone cortex and an illuminating device having an outer diameter of 6.0 mm is preferably placed in the cannula having the outer diameter of 7.0 mm. In this particular embodiment, the 6.0 mm outer diameter illuminating device has no open door space. The 7.0 mm, 10.0 mm, and the 13.0 mm outer diameter cannula will have no flared portion at the proximal end thereof and will also be available with an optional handle attachment having a threaded screw head of 0.8 mm in depth that matches the threaded recessed portion at the proximal end of the cannula. In one embodiment, the cannula and their corresponding illuminating devices preferably have different lengths of between 10 cm-55 cm in 15 cm increments.

In one embodiment, the illuminating device preferably includes direct fiber optic-assisted or LED assisted visualization to help confirm adequate position of the ultimate cannula where it engages the bone cortex. The light source may be removed while the outer cylinder is left in place engaging the outer bone cortex. The drill for the corresponding lag screw, proximal locking screw and/or distal locking screw may be placed through the drill guide that is positioned in the final cannula. The bone cortex may be drilled along the proposed track of the corresponding screw using fluoroscopic guidance. The drill guide and the drill are preferably removed while keeping the cannula in place engaging the bone cortex. The illuminating device may be placed in the cannula and a depth gauge may be advanced into the drill hole and the length of the drill hole and a corresponding screw length determined via direct visual and fluoroscopic assistance. A bone screw having sufficient length may be placed under direct visualization. The illuminating device and the cannula may be removed. In one embodiment, an end cap may be placed in the base of the intramedullary nail.

In one embodiment, the guide wires may be made of solid, sterile, stainless steel individually packaged. The guide wires may be provided in various lengths and diameters having either a sharp, smooth, pointed tip or a terminally threaded tip with outer threads having an equal outer diameter as the smooth portion of the shaft of the guide wire. Other sterile materials having required mechanical properties of strength and inertness may also be used for the guide wire.

The tissue dilators are preferably made of sterile, plastic polymers provided in sterile packages. The tissue dilators may be provided as a set whereby each of the individual tissue dilators has a unique diameter and/or length. The tissue dilators may also be made of other sterile materials including sterile, autoclaveable metal or carbon fiber combinations having the required mechanical properties of radiolucency, strength and inertness.

In one embodiment, the cannula may be made of sterile, autoclavable, carbon fiber of varying diameters, lengths and/or shapes. The cannula may also be made of other materials having mechanical properties of strength and inertness.

In one embodiment, the illuminating device may be made of sterilizable, transparent plastic polymers that are able to withstand the heat generated by light generating elements such fiber optic illuminated light sources without deforming the shape of the illuminating device. In other embodiments, the illuminating device may be made of other sterilizable and/or sterile, package materials having the required mechanical properties of strength and inertness.

The minimally invasive endoscopic system disclosed herein may also be used for the placement of other orthopedic surgical implants including, but not limited to, plate osteosynthesis for open reduction and internal fixation, percutaneous pinning, schantz screw and pin placement for external fixation applications, and spinal stabilization with pedicel screw and rod placement.

The various components shown in the drawing figures are not drawn to scale and the actual measurements of the devices illustrated in this invention may constitute dimensions other than those specified herein.

While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, or incorporated by reference herein, may be incorporated with any of the features shown in any of the other embodiments described herein, or incorporated by reference herein, and still fall within the scope of the present invention.

### STATEMENT OF INDUSTRIAL APPLICABILITY

The present invention has applicability in the medical industry.

What is claimed is:

1. A system for percutaneously placing intramedullary nails comprising:
a guide wire percutaneously insertable into tissue for forming an opening in the tissue and engaging a cortex of a bone;
at least one tissue dilator slideable over an outer surface of said guide wire for widening the tissue opening formed by said guide wire;
a cannula slideable over an outer surface of said at least one tissue dilator for further widening the tissue opening and providing access to the cortex of the bone, said cannula having an outer wall extending between proximal and distal ends thereof, said outer wall having a smaller portion including a door that extends between the proximal end distal ends of said cannula and a larger portion that bounds said door, wherein said door is selectively detachable from the larger portion of said outer wall for opening an elongated slot in said outer wall that extends from the proximal end to the distal end of said cannula.

2. The system as claimed in claim 1, wherein said outer wall has an inner surface and an outer surface, and wherein said elongated slot in said outer wall extends from the inner surface of said outer wall to the outer surface of said outer wall.

3. The system as claimed in claim 1, wherein said outer wall of said cannula has a cylindrical shape.

4. The system as claimed in claim 1, wherein said door is slideable toward the distal end of said cannula for closing said elongated slot in said outer wall and is slideable toward the proximal end of said cannula for opening said elongated slot in said outer wall.

5. The system as claimed in claim 4, further comprising a locking system having a locked state that prevents said door from sliding toward the proximal end of said cannula and an unlocked state that releases said door for sliding toward the proximal end of said cannula.

6. The system as claimed in claim 5, wherein said locking system comprises a moveable locking element adapted to engage said sliding door when in the locked state and be disengaged from said sliding door in the unlocked state.

7. The system as claimed in claim 1, further comprising:
an intramedullary nail insertable into said cannula for passing through the tissue opening and engaging the cortex of the bone; and
an alignment device coupled with said intramedullary nail.

8. The system as claimed in claim 7, wherein while said intramedullary nail engages the bone, said cannula is moveable away from said intramedullary nail by detaching said door from said cannula to open said elongated slot and passing said elongated slot over said intramedullary nail.

9. The system as claimed in claim 1, wherein said guide wire has a distal end including a pointed tip adapted to pierce through skin, tissue and bone.

10. The system as claimed in claim 9, wherein the distal end of said guide wire comprises threads.

11. The system as claimed in claim 1, wherein said at least one tissue dilator comprises a plurality of cannulated tissue dilators having progressively larger diameters, wherein a first one of said cannulated tissue dilators has an inner diameter that is slightly larger than an outer diameter of said guide wire and a second one of said cannulated tissue dilators has an inner diameter that is slightly larger than an outer diameter of said first cannulated tissue dilator.

12. The system as claimed in claim 1, wherein each said tissue dilator tapers outwardly between distal and proximal ends thereof so that outer diameters of said tissue dilators at the distal ends thereof are smaller than outer diameters of said tissue dilators at the proximal ends thereof.

13. The system as claimed in claim 1, further comprising a cannulated illuminating device insertable into said cannula for illuminating the cortex of the bone.

14. The system as claimed in claim 13, wherein said cannulated illuminating device comprises:
an outer wall having a proximal end and a distal end;
light emitting elements adapted to project light from the distal end of said outer wall;
an elongated opening in said outer wall of said illuminating device that extends from the proximal end to the distal end of said outer wall of said illuminating device.

15. The system as claimed in claim 14, wherein a cross-section of said outer wall of said cannulated illuminating device defines a C shape.

16. The system as claimed in claim 15, wherein said cannulated illuminating device is insertable into said cannula so that said elongated opening in said outer wall of said cannulated illuminating device is substantially aligned with said elongated slot in said outer wall of said cannula.

17. A system for percutaneously placing intramedullary nails comprising:
a guide wire for forming a percutaneous opening in tissue and engaging a cortex of a bone;
at least one tissue dilator advanceable over said guide wire for widening the tissue opening;
a cannula advanceable over said at least one tissue dilator for further widening the tissue opening and providing access to the cortex of the bone, said cannula having an outer wall extending between proximal and distal ends thereof, said outer wall including an elongated slot extending from the proximal end to the distal end of said cannula and a door for closing said elongated slot, wherein said door is selectively detachable from said outer wall for opening said elongated slot.

18. The system as claimed in claim 17, further comprising an intramedullary nail insertable into said cannula for engaging the cortex of the bone, wherein said door is adapted to be detached from said outer wall of said cannula to open said elongated slot so that said intramedullary nail may pass through said elongated slot for removing said cannula from a surgical site as said intramedullary nail remains in contact with the cortex of the bone.

19. The system as claimed in claim 17, further comprising a cannulated illuminating device insertable into said cannula for providing illumination at the cortex of the bone, wherein said illuminating device includes an outer wall having an elongated opening extending from the proximal end to the distal end thereof, wherein said elongated opening of said outer wall of said illuminating device is substantially aligned with said elongated slot of said cannula.

20. A system for percutaneously placing intramedullary nails comprising:
a cannula insertable into a percutaneous tissue opening for providing access to a cortex of a bone, said cannula having an outer wall extending between proximal and distal ends thereof, said outer wall having a smaller portion including a door that extends between the proximal and distal ends of said cannula and a larger portion that bounds said door, wherein said door is selectively detachable from the larger portion of said outer wall for opening an elongated slot in said outer wall that extends from the proximal end to the distal end of said cannula.

21. The system as claimed in claim 20, further comprising an intramedullary nail insertable into said cannula for engaging the cortex of the bone, wherein while said intramedullary nail engages the bone, said cannula is moveable away from said intramedullary nail by detaching said door from said cannula and passing said elongated slot of said cannula over said intramedullary nail.

22. The system as claimed in claim 20, further comprising:
a guide wire insertable into the tissue for initially forming the percutaneous tissue opening and engaging the cortex of the bone;
at least one tissue dilator slideable over an outer surface of said guide wire for widening the tissue percutaneous tissue opening initially formed by said guide wire and engaging the cortex of the bone, wherein said cannula is slideable over an outer surface of said at least one tissue dilator for further widening the percutaneous tissue opening and providing access to the cortex of the bone.
